(19) United States
(12) Patent Application Publication
(10) Pub. No.: US 2023/0070906 A1
(43) Pub. Date: Mar. 9, 2023

(54) INFORMATION PROCESSING APPARATUS, METHOD, AND PROGRAM

(71) Applicant: FUJIFILM Corporation, Tokyo (JP)

(72) Inventor: Akimichi ICHINOSE, Tokyo (JP)

(73) Assignee: FUJIFILM Corporation, Tokyo (JP)

(21) Appl. No.: 17/897,195

(22) Filed: Aug. 28, 2022

(30) Foreign Application Priority Data
Sep. 6, 2021 (JP) 2021-144650

(51) Int. Cl. G06N 20/00; G06F 40/30; G06F 40/20

(52) U.S. Cl. CPC G06N 20/00; G06F 40/30; G06F 40/20

(57) ABSTRACT

The processor divides a sentence into predetermined units, determines at least one of an attribute, a factuality, or a relationship of each unit, decides a weight for each unit according to a result of the determination, and derives a feature amount of each unit by using a derivation model constructed by machine learning and derive a feature amount of the sentence by performing weighting calculation on the feature amount of each unit based on the weight.

### CROSS REFERENCE TO RELATED APPLICATIONS

The present application claims priority under 35 U.S.C. § 119 to Japanese Patent Application No. 2021-144650 filed on Sep. 6, 2021. The above application is hereby expressly incorporated by reference, in its entirety, into the present application.

### BACKGROUND

### Technical Field

The present disclosure relates to an information processing apparatus, method, and program.

### Related Art

Various methods for analyzing sentences have been proposed. For example, JP2016-151827A proposes a method of structuring and presenting terms belonging to categories such as a part name, a disease name, and a size by analyzing a plurality of terms extracted from an interpretation report. Further, JP2020-038602A proposes a method of creating a summary of medical records using importance information indicating the importance of each element generated based on elements constituting a medical record sentence obtained by performing text analysis on the medical record sentence, and a summary sentence corresponding to the medical record sentence.

In addition, a method has been proposed in which a feature amount of a sentence is derived by analyzing the sentence and various inferences are made based on the feature amount. For example, a method has been proposed in which a feature amount of a sentence is derived by analyzing the sentence and a task of classifying the sentence is performed based on the feature amount (see Hierarchical Attention Networks for Document Classification, Zichao Yang et al., 2016 Conference of the North American Chapter of the Association for Computational Linguistics: Human Language Technologies, NAACL HLT 2016—Proceedings of the Conference (2016) 1480-1489). In the method as described in Hierarchical Attention Networks for Document Classification, Zichao Yang et al., 2016 Conference of the North American Chapter of the Association for Computational Linguistics: Human Language Technologies, NAACL HLT 2016—Proceedings of the Conference (2016) 1480-1489), an analysis model constructed by machine learning a neural network using supervised training data in which the sentence and the classification result are associated is used.

A large amount of supervised training data is required to derive the feature amount of the sentence so that various tasks can be performed accurately. However, since the number of sentences is limited, it is difficult to prepare a large amount of supervised training data. Therefore, it is difficult to construct an analysis model that can accurately derive a feature amount of a sentence.

### SUMMARY OF THE INVENTION

The present disclosure has been made in view of the above circumstances, and an object thereof is to enable accurate derivation of a feature amount of a sentence without preparing a large amount of supervised training data.

According to an aspect of the present disclosure, there is provided an information processing apparatus comprising at least one processor, in which the processor is configured to divide a sentence into predetermined units, determine at least one of an attribute, a factuality, or a relationship of each unit, decide a weight for each unit according to a result of the determination, and derive a feature amount of each unit by using a derivation model constructed by machine learning and derive a feature amount of the sentence by performing weighting calculation on the feature amount of each unit based on the weight.

In the information processing apparatus according to the aspect of the present disclosure, the processor may be configured to decide a weight so that a weight for a word determined by a predetermined attribute, factuality, and relationship is greater than a weight for a word determined by an attribute, factuality, and relationship other than the predetermined attribute, factuality, and relationship.

In the information processing apparatus according to the aspect of the present disclosure, the predetermined attribute, factuality, and relationship may be decided according to a task using the derived feature amount of the sentence.

In the information processing apparatus according to the aspect of the present disclosure, the processor may be configured to decide the weight by using the derivation model.

In the information processing apparatus according to the aspect of the present disclosure, the processor may be configured to display the sentence by emphasizing words determined by a predetermined attribute, factuality, and relationship.

In the information processing apparatus according to the aspect of the present disclosure, the processor may be configured to perform a task of specifying a content of the sentence by using the derived feature amount.

In the information processing apparatus according to the aspect of the present disclosure, the processor may be configured to perform a task of searching for an image corresponding to the sentence by using the derived feature amount.

According to another aspect of the present disclosure, there is provided an information processing method comprising: dividing a sentence into predetermined units; determining at least one of an attribute, a factuality, or a relationship of each unit; deciding a weight for each unit according to a result of the determination; and deriving a feature amount of each unit by using a derivation model constructed by machine learning and deriving a feature amount of the sentence by performing weighting calculation on the feature amount of each unit based on the weight.

In addition, a program for causing a computer to execute the information processing method according to the aspect of the present disclosure may be provided.

According to the aspects of the present disclosure, it is possible to accurately derive the feature amount of a sentence without preparing a large amount of supervised training data.

### BRIEF DESCRIPTION OF THE DRAWINGS

FIG. 1 is a diagram showing a schematic configuration of a medical information system to which an information processing apparatus according to a first embodiment of the present disclosure is applied.

FIG. 2 is a diagram showing a schematic configuration of the information processing apparatus according to the first embodiment.

FIG. 3 is a functional configuration diagram of the information processing apparatus according to the first embodiment.

FIG. 4 is a diagram showing a creation screen of an interpretation report in the first embodiment.

FIG. 5 is a diagram for describing the determination of attributes and factuality in the first embodiment.

FIG. 6 is a diagram schematically showing a derivation model.

FIG. 7 is a diagram schematically showing another example of a derivation model.

FIG. 8 is a diagram showing a creation screen of an interpretation report in which a specific result of a disease name is displayed.

FIG. 9 is a diagram showing the creation screen of the interpretation report in which the specific result of the disease name is displayed.

FIG. 10 is a flowchart showing a process performed in the first embodiment.

FIG. 11 is a functional configuration diagram of an information processing apparatus according to a second embodiment.

FIG. 12 is a diagram for describing a search performed in the information processing apparatus according to the second embodiment.

FIG. 13 is a diagram showing a creation screen of an interpretation report in the second embodiment.

FIG. 14 is a flowchart showing a process performed in the second embodiment.

FIG. 15 is a diagram for describing the determination of an attribute, factuality, and relationship in a third embodiment.

FIG. 16 is a diagram schematically showing weighting in a derivation model in the third embodiment.

### DETAILED DESCRIPTION

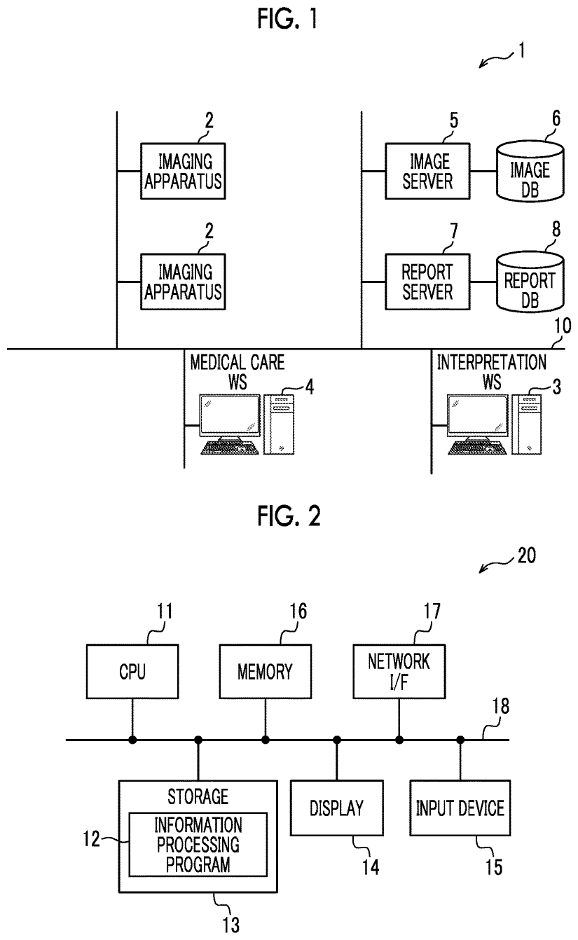

Hereinafter, embodiments of the present disclosure will be described with reference to the drawings. First, a configuration of a medical information system 1 to which an information processing apparatus according to a first embodiment is applied will be described. FIG. 1 is a diagram showing a schematic configuration of the medical information system 1. The medical information system 1 shown in FIG. 1 is, based on an examination order from a doctor in a medical department using a known ordering system, a system for imaging an examination target part of a subject, storing a medical image acquired by the imaging, interpreting the medical image by a radiologist and creating an interpretation report, and viewing the interpretation report and observing the medical image to be interpreted in detail by the doctor in the medical department that is a request source.

Each apparatus is a computer on which an application program for causing each apparatus to function as a component of the medical information system 1 is installed. The application program is stored in a storage apparatus of a server computer connected to the network 10 or in a network storage in a state in which it can be accessed from the outside, and is downloaded to and installed on the computer in response to a request. Alternatively, the application program is recorded on a recording medium, such as a digital versatile disc (DVD) and a compact disc read only memory (CD-ROM), and distributed, and is installed on the computer from the recording medium.

The imaging apparatus 2 is an apparatus (modality) that generates a medical image showing a diagnosis target part of the subject by imaging the diagnosis target part. Specifically, it is a simple X-ray imaging apparatus, a computed tomography (CT) apparatus, a magnetic resonance imaging (MRI) apparatus, a positron emission tomography (PET) apparatus, and the like. The medical image generated by the imaging apparatus 2 is transmitted to the image server 5 and is saved in the image DB 6.

The interpretation WS 3 is a computer used by, for example, a radiologist of a radiology department to interpret a medical image and to create an interpretation report, and encompasses an information processing apparatus 20 according to the present embodiment. In the interpretation WS 3, a viewing request for a medical image to the image server 5, various image processing for the medical image received from the image server 5, display of the medical image, input reception of comments on findings regarding the medical image, and the like are performed. In the interpretation WS 3, an analysis process for medical images and input comments on findings, support for creating an interpretation report based on the analysis result, a registration request and a viewing request for the interpretation report to the report server 7, and display of the interpretation report received from the report server 7 are performed. The above processes are performed by the interpretation WS 3 executing software programs for respective processes.

The medical care WS 4 is a computer used by a doctor in a medical department to observe an image in detail, view an interpretation report, create an electronic medical record, and the like, and is configured to include a processing apparatus, a display apparatus such as a display, and an input apparatus such as a keyboard and a mouse. In the medical care WS 4, a viewing request for the image to the image server 5, display of the image received from the image server 5, a viewing request for the interpretation report to the report server 7, and display of the interpretation report received from the report server 7 are performed. The above processes are performed by the medical care WS 4 executing software programs for respective processes.

The image server 5 is a general-purpose computer on which a software program that provides a function of a database management system (DBMS) is installed. The image server 5 comprises a storage in which the image DB 6 is configured. This storage may be a hard disk apparatus connected to the image server 5 by a data bus, or may be a disk apparatus connected to a storage area network (SAN) or a network attached storage (NAS) connected to the network 10. In a case where the image server 5 receives a request to register a medical image from the imaging apparatus 2, the image server 5 prepares the medical image in a format for a database and registers the medical image in the image DB 6.

Image data of the medical image acquired by the imaging apparatus 2 and accessory information are registered in the image DB 6. The accessory information includes, for example, an image identification (ID) for identifying each medical image, a patient ID for identifying a subject, an examination ID for identifying an examination, a unique ID (unique identification (UID)) allocated for each medical image, examination date and examination time at which a medical image is generated, the type of imaging apparatus used in an examination for acquiring a medical image, patient information such as the name, age, and gender of a patient, an examination part (an imaging part), imaging information (an imaging protocol, an imaging sequence, an imaging method, imaging conditions, the use of a contrast medium, and the like), and information such as a series number or a collection number in a case where a plurality of medical images are acquired in one examination.

In addition, in a case where the viewing request from the interpretation WS 3 and the medical care WS 4 is received through the network 10, the image server 5 searches for a medical image registered in the image DB 6 and transmits the searched for medical image to the interpretation WS 3 and to the medical care WS 4 that are request sources.

The report server 7 incorporates a software program for providing a function of a database management system to a general-purpose computer. In a case where the report server 7 receives a request to register the interpretation report from the interpretation WS 3, the report server 7 prepares the interpretation report in a format for a database and registers the interpretation report in the report DB 8.

In the report DB 8, an interpretation report including at least the comments on findings created in the interpretation WS 3 is registered. The interpretation report may include, for example, information such as a medical image to be interpreted, an image ID for identifying the medical image, a radiologist ID for identifying the radiologist who performed the interpretation, a lesion name, lesion position information, information for accessing a medical image including a specific region, and property information.

Further, in a case where the report server 7 receives the viewing request for the interpretation report from the interpretation WS 3 and the medical care WS 4 through the network 10, the report server 7 searches for the interpretation report registered in the report DB 8, and transmits the searched for interpretation report to the interpretation WS 3 and to the medical care WS 4 that are request sources.

The medical image is not limited to the CT image, and any medical image such as an MRI image and a simple two-dimensional image acquired by a simple X-ray imaging apparatus can be used.

The network 10 is a wired or wireless local area network that connects various apparatuses in a hospital to each other. In a case where the interpretation WS 3 is installed in another hospital or clinic, the network 10 may be configured to connect local area networks of respective hospitals through the Internet or a dedicated line.

Next, the information processing apparatus according to the first embodiment will be described. FIG. 2 describes a hardware configuration of the information processing apparatus according to the first embodiment. As shown in FIG. 2, the information processing apparatus 20 includes a central processing unit (CPU) 11, a non-volatile storage 13, and a memory 16 as a temporary storage area. Further, the information processing apparatus 20 includes a display 14 such as a liquid crystal display, an input device 15 such as a keyboard and a mouse, and a network interface (I/F) 17 connected to the network 10. The CPU 11, the storage 13, the display 14, the input device 15, the memory 16, and the network I/F 17 are connected to a bus 18. The CPU 11 is an example of a processor in the present disclosure.

The storage 13 is realized by a hard disk drive (HDD), a solid state drive (SSD), a flash memory, and the like. An information processing program is stored in the storage 13 as the storage medium. The CPU 11 reads the information processing program 12 from the storage 13, loads the read program into the memory 16, and executes the loaded information processing program 12.

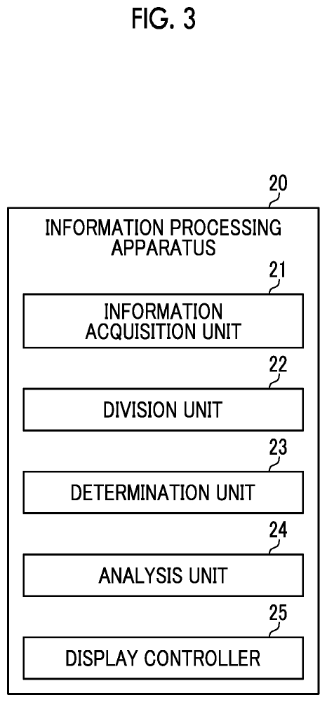

Next, a functional configuration of the information processing apparatus according to the first embodiment will be described. FIG. 3 is a diagram showing a functional configuration of the information processing apparatus according to the first embodiment. As shown in FIG. 3, the information processing apparatus 20 comprises an information acquisition unit 21, a division unit 22, a determination unit 23, an analysis unit 24, and a display controller 25. Then, in a case where the CPU 11 executes the information processing program 12, the CPU 11 functions as the information acquisition unit 21, the division unit 22, the determination unit 23, the analysis unit 24, and the display controller 25. The information processing apparatus according to the present embodiment performs a task of specifying a disease name represented by comments on findings created by a radiologist. The task of specifying the disease name is an example of a task of specifying the content of a sentence.

The information acquisition unit 21 acquires a target medical image GO for creating an interpretation report from the image server 5 according to an instruction from the input device 15 by the radiologist who is an operator. The target medical image GO is displayed on the display 14 via the display controller 25. Specifically, the target medical image GO is displayed on the display 14 on the creation screen of the interpretation report.

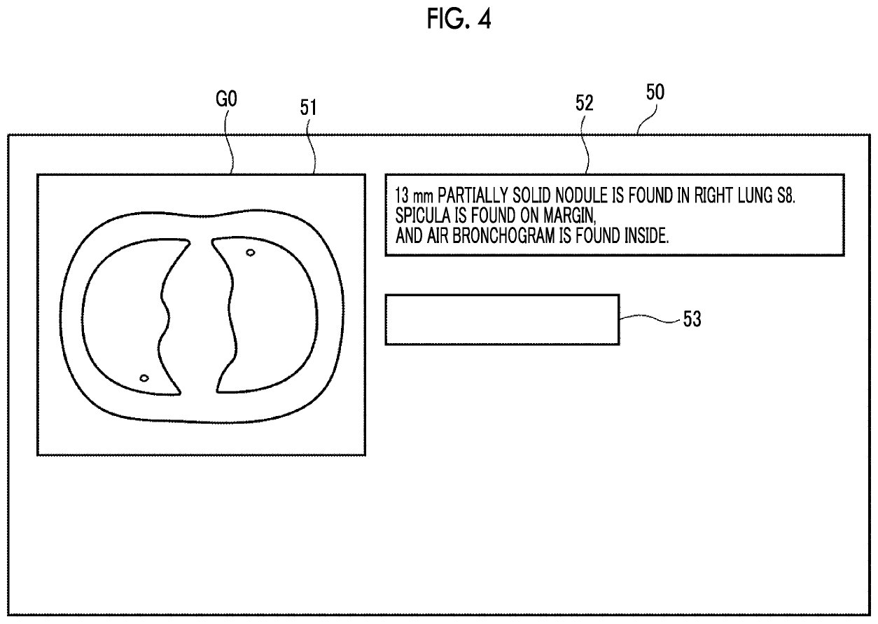

FIG. 4 is a diagram showing a creation screen of an interpretation report. As shown in FIG. 4, a creation screen 50 includes an image display region 51, a sentence display region 52, and a specific result display region 53. The target medical image GO acquired by the information acquisition unit 21 is displayed in the image display region 51. In FIG. 4, the target medical image GO is one tomographic image constituting a three-dimensional image of a chest. In the sentence display region 52, the comments on findings input by a doctor are displayed. As shown in FIG. 4, the comments on findings are “A 13 mm partially solid nodule is found in a right lung S8. A spicula is found on the margin, and an air bronchogram is found inside.” The specific result display region 53 will be described later.

The division unit 22 divides the comments on findings into predetermined units. Specifically, the comments on findings are divided into words by morphological analysis of the comments on findings. Thereby, the comments on findings displayed in the sentence display region 52 are divided into the words “A 13 mm/partially solid/nodule/is/found/in/a right lung/S8/. A spicula/is/found/on/the margin/,/and/an air bronchogram/is/found/inside/.” The division unit 22 may divide the comments on findings into clauses.

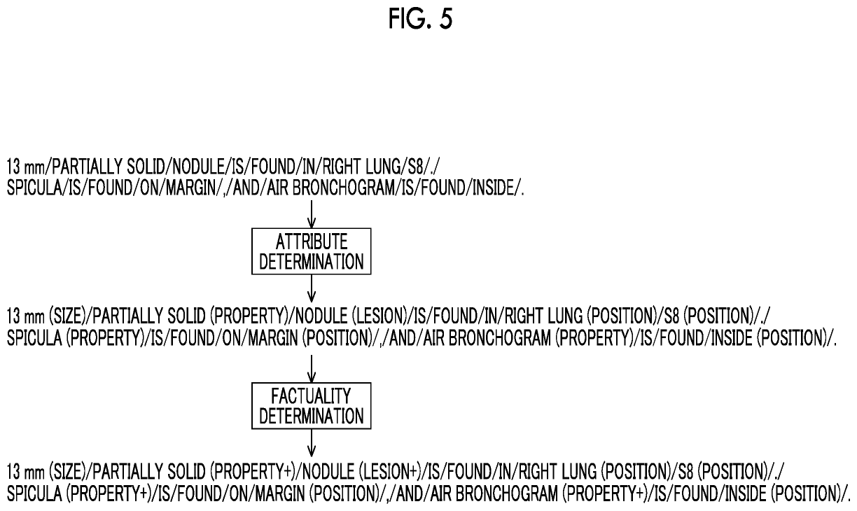

The determination unit 23 determines at least one of the attribute, factuality, or relationship of each divided word. In the present embodiment, the determination unit 23 determines the attribute and factuality of each divided word. FIG. 5 is a diagram for describing the determination of attributes and factuality. With respect to the attributes, the determination unit 23 determines, for example, which of the attributes of position, size, property, lesion, change, and disease name the word has. For example, with respect to the above comments on findings, the determination unit 23 determines that “right lung”, “S8”, “margin”, and “inside” have the position attribute, “13 mm” has the size attribute, “partially solid”, “spicula”, and “air bronchogram” have the property attribute, and “nodule” has the lesion attribute. The types of attributes are not limited to the position, size, property, lesion, change, and disease name.

With respect to the factuality, the determination unit 23 determines whether the attributes of the property, lesion, and disease name indicate negative, positive, or suspicious. Here, in the above comments on findings, the words determined to have the attributes of the property, lesion, and disease name are “partially solid”, “nodule”, “spicula”, and “air bronchogram”. All of these words are positive because the context ends with “found”. Therefore, the determination unit 23 determines that all the factualities of the words “partially solid”, “nodule”, “spicula”, and “air bronchogram” are positive. In FIG. 5, the positive sign is indicated by adding a + sign after the attribute. In a case where the result is negative, a − sign is added, and in a case where there is a suspicion, a ± sign may be added.

The analysis unit 24 derives the feature amount of each word, decides the weight for each word according to the result of the determination by the determination unit 23, and performs weighting calculation on the feature amount for each word based on the decided weight, thereby deriving the feature amount of the comments on findings. To this end, the analysis unit 24 derives the feature amount of the comments on findings by using the derivation model constructed by machine learning the neural network.

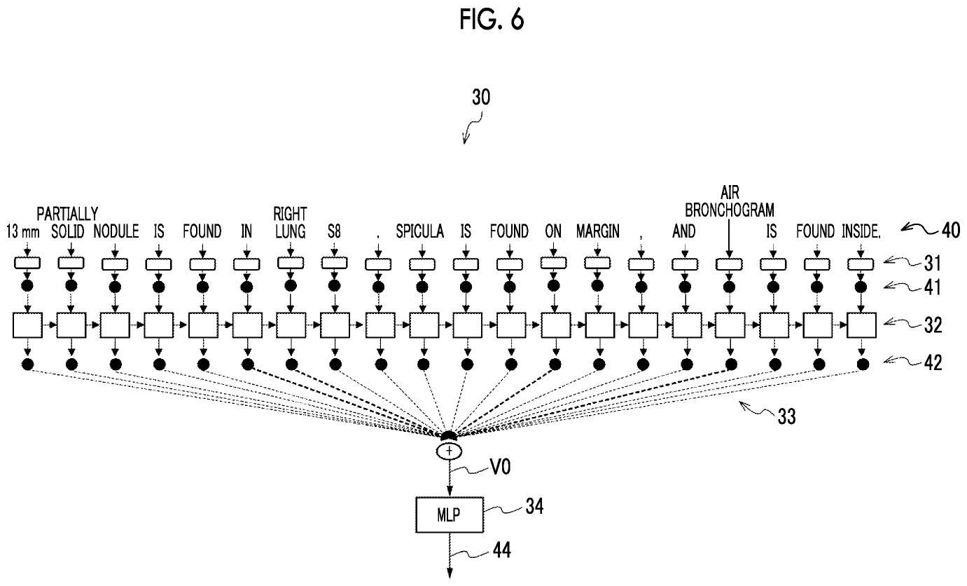

FIG. 6 is a diagram schematically showing a derivation model. As shown in FIG. 6, a derivation model 30 includes an embedding layer 31, a recurrent neural network layer (hereinafter referred to as an RNN layer) 32, a weighting calculation mechanism 33, and a multi-layer perceptron (MLP) 34. The analysis unit 24 inputs each word 40 derived by the division unit 22 by dividing the comments on findings into the embedding layer 31. The embedding layer 31 outputs a feature vector 41 of each word 40. In FIG. 6, the feature vector is indicated by a black circle. The feature vector 41 of each word 40 is an n-dimensional vector. The RNN layer 32 outputs a feature vector 42 of each word 40 in consideration of the context of the feature vector 41 output by the embedding layer 31. The weighting calculation mechanism 33 performs weighting calculation on the feature vector 42 of each word 40 to derive the feature vector of the comments on findings input to the analysis unit 24 as a feature amount V0. The feature amount V0 is also an n-dimensional vector. The MLP 34 outputs the disease name represented by the comments on findings input from the feature amount V0 as a specific result 44.

The weighting calculation mechanism 33 decides the weight in the case where weighting calculation on the feature vector 42 of each word 40 is performed. In the present embodiment, the weighting calculation mechanism 33 decides a weight so that a weight for a word determined by the predetermined attribute and factuality is greater than a weight for a word determined by an attribute and factuality other than the predetermined attribute and factuality. The predetermined attribute can be, for example, properties and disease names. In addition, the predetermined factuality can be positive.

Therefore, the weighting calculation mechanism 33 decides weights so that weights for the “partially solid”, “nodule”, “spicula”, and “air bronchogram” are greater than the weights for the other words. In FIG. 6, by making the arrows in the weighting calculation mechanism 33 of the feature vectors 42 of the “partially solid”, “nodule”, “spicula” and “air bronchogram” output by the RNN layer 32 thicker than the other arrows, it is shown that the weights for the “partially solid”, “nodule”, “spicula”, and “air bronchogram” are greater than the weights for other words. In FIG. 6, by making “partially solid”, “nodule”, “spicula”, and “air bronchogram” among the words 40 input to the embedding layer 31 bold, it is also shown that the weights of these words are greater than those of the other words.

In the present embodiment, the total weight is 1. The weighting calculation mechanism 33 decides the weights so that, for example, 80% of the weights are assigned to a predetermined attribute and factuality. In the present embodiment, the comments on findings are divided into 20 words by the division unit 22, and of these, the words determined by the predetermined attribute and factuality are four words, “partially solid”, “nodule”, “spicula”, and “air bronchogram”. Therefore, the weighting calculation mechanism 33 decides the weight for each of the four words to be 0.8/4=0.2. In addition, the weights for words other than the “partially solid”, “nodule”, “spicula”, and “air bronchogram” are decided to be 0.2/16=0.0125.

Then, the weighting calculation mechanism 33 derives the feature amount V0 by weighting and adding all the feature vectors 42 by the decided weights.

In the present embodiment, the analysis unit 24 performs a task of specifying the disease name in the comments on findings using the feature amount V0. Therefore, it may be decided that the weights of attributes and factuality that are less relevant to the task are small. For example, the attributes required to specify a disease name are properties and lesions. For this reason, the weights for words whose properties and lesions are positive attributes may be greater than the weights for other words.

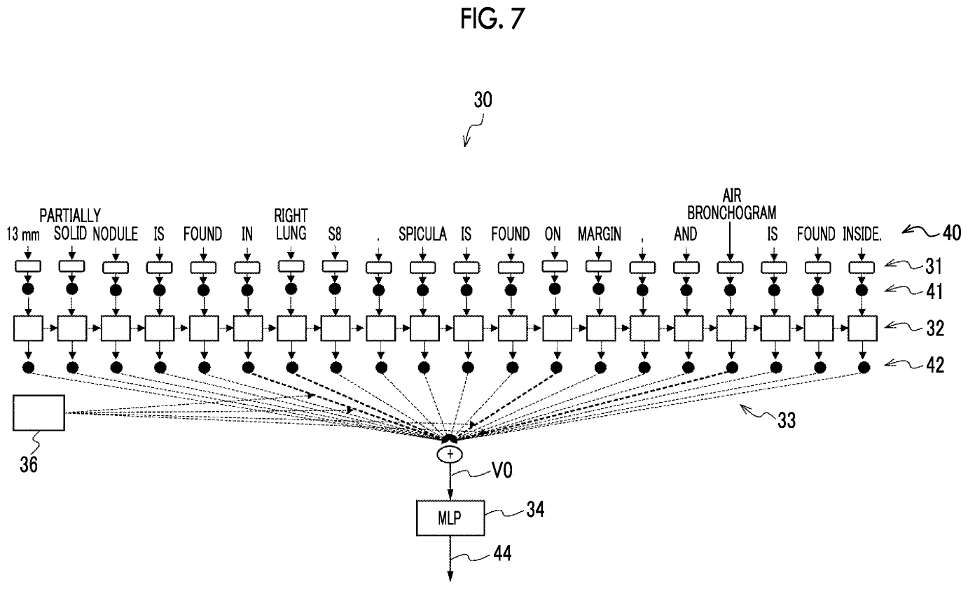

On the other hand, as shown in FIG. 7, the weighting calculation mechanism 33 may be provided with a context vector 36 that has been trained to increase the weight for a predetermined attribute and factuality. The context vector 36 is trained so that the attribute and factuality that contribute more to the derivation of the feature amount V0, that is, the predetermined attribute and factuality are given a greater weight. Then, the context vector 36 outputs a vector in which a greater weight is obtained as much as the predetermined attribute and factuality. The weighting calculation mechanism 33 decides the inner product of the vector derived from the context vector 36 and each feature vector 42 as a weight for each feature vector 42.

In addition, although FIG. 7 shows a state in which the weight from the context vector 36 is applied by giving arrows only to the feature vectors 42 of the “partially solid”, “nodule”, “spicula”, and “air bronchogram” in which the weight of the context vector 36 is increased, the weight from the context vector 36 is also applied to the feature vectors 42 of other words.

The context vector 36 may be constructed by learning so that the weights of attributes and factuality that are less relevant to the task are small.

The MLP 34 is a fully connected neural network, and in a case where the feature amount V0 is input, it is constructed by machine learning the fully connected neural network so as to specify the disease name represented by the comments on findings. The disease name specified by the MLP 34 also includes benign.

The MLP 34 outputs scores for a plurality of types of lung diseases, specifies the disease having the highest output score as a disease related to the comments on findings, and outputs a specific result 44. For example, in a case where the MLP 34 is trained to specify three disease names, “benign”, “lung adenocarcinoma”, and “squamous cell carcinoma”, the MLP 34 outputs scores for “benign”, “lung adenocarcinoma”, and “squamous cell carcinoma”. Then, the MLP 34 specifies the disease name having the maximum score as the disease name in the comments on findings. For example, in a case where the scores for “benign”, “lung adenocarcinoma”, and “squamous cell carcinoma” are 0.1, 0.8, and 0.1, respectively, “lung adenocarcinoma” is specified as the disease name, and the specific result 44 is output.

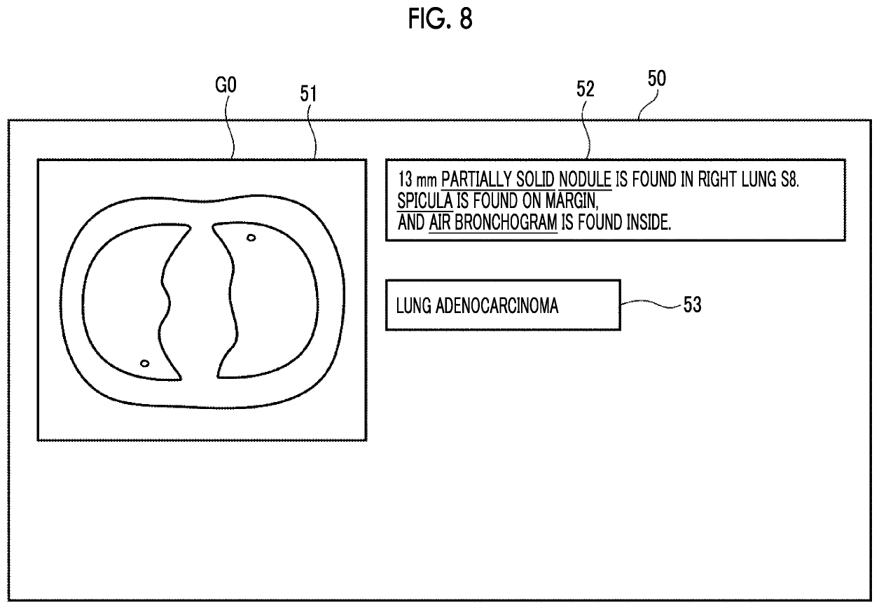

In addition to the target medical image GO and the comments on findings, the display controller 25 further displays the specific result of the disease name on the creation screen of the interpretation report. FIG. 8 is a diagram showing a creation screen of an interpretation report in which a specific result of a disease name is displayed. As shown in FIG. 8, the display controller 25 displays the disease name represented by the comments on findings specified by the analysis unit 24 in the specific result display region 53. In FIG. 8, the disease name is “lung adenocarcinoma”.

Further, the display controller 25 may emphasize a word determined by a predetermined attribute and factuality in the comments on findings displayed in the sentence display region 52. In FIG. 8, the “partially solid”, “nodule”, “spicula”, and “air bronchogram” are highlighted by underlining each of these words. The highlighting is not limited to the addition of underlining, and the highlighting may be performed by emphasizing characters, adding markers to words, or the like.

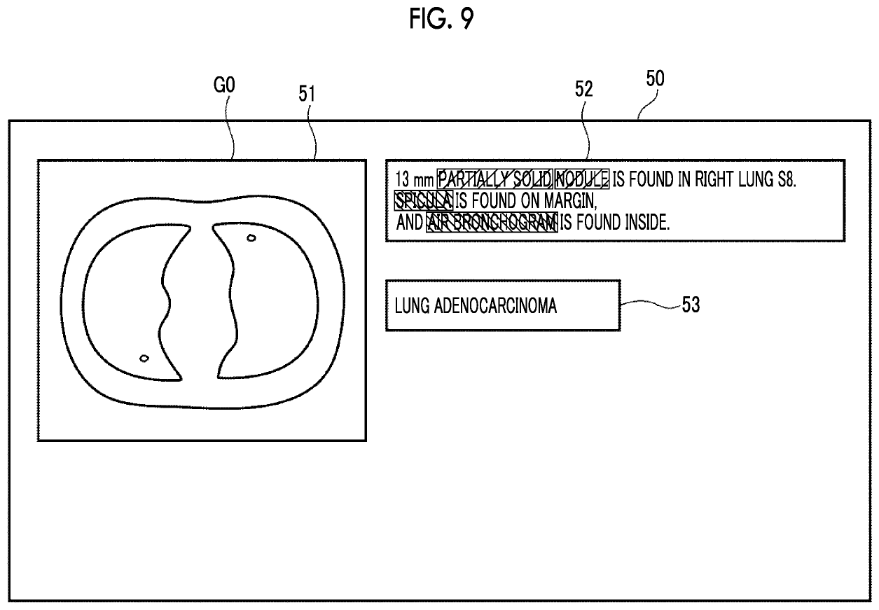

Further, in a case where weighting is performed using the context vector 36, the degree of emphasis of the word may be different depending on the size of the vector output by the context vector 36. For example, it assumed that the size of the vector output by the context vector 36 for each of “partially solid”, “nodule”, “spicula”, and “air bronchogram” is “partially solid”=“nodule”<“spicula”=“air bronchogram”. In this case, as shown in FIG. 9, the degree of emphasis on the words “spicula” and “air bronchogram” may be greater than the degree of emphasis on “partially solid” and “nodule” in the comments on findings. In addition, in FIG. 9, the difference in the degree of emphasis is shown by the difference in the spacing of the hatch lines.

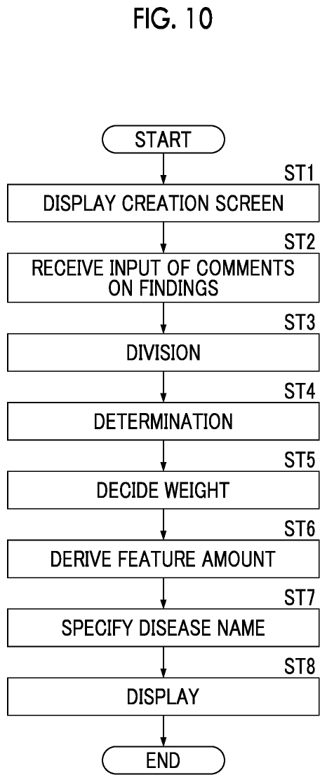

Next, a process performed in the first embodiment will be described. FIG. 10 is a flowchart showing a process performed in the first embodiment. It is assumed that the target medical image GO is acquired from the image server 5 by the information acquisition unit 21 and is saved in the storage 13.

First, the display controller 25 displays the creation screen of the interpretation report (Step ST1) and receives the input of the comments on findings (Step ST2). Next, the division unit 22 divides the comments on findings into words (Step ST3), and the determination unit 23 determines at least one of the attribute, factuality, or relationship of each divided word (Step ST4).

Then, the weighting calculation mechanism 33 of the analysis unit 24 decides the weight for each word according to the determination result (Step ST5), and performs weighting calculation on the feature amount for each word based on the decided weight, thereby deriving the feature amount V0 of the comments on findings (Step ST6). Further, the MLP 34 of the analysis unit 24 specifies the disease name represented by the comments on findings based on the feature amount V0 (Step ST7). Then, the display controller displays the specified disease name on the creation screen 50 of the interpretation report (Step ST8), and ends the process.

As described above, in the present embodiment, the comments on findings are divided into, for example, predetermined units such as words, at least one of the attribute, factuality, or relationship of each unit is determined, the weight for each unit is decided according to the determination result, and weighting calculation on the feature amount of each unit is performed based on the decided weight to derive the feature amount of the comments on findings. Therefore, it is possible to derive a feature amount V0 effective for a task such as specifying a disease name represented by comments on findings without constructing the derivation model 30 using a large amount of supervised training data.

Further, by displaying the weight decided by the weighting calculation mechanism 33, the unit used as the basis for deriving the feature amount can be easily checked.

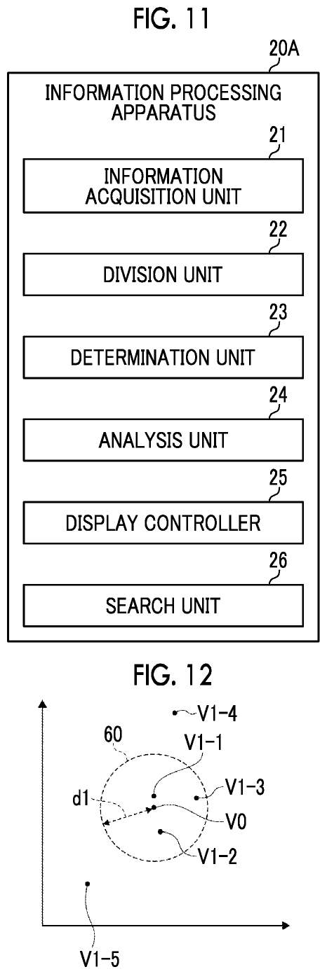

Next, a second embodiment of the information processing apparatus according to the present disclosure will be described. FIG. 11 is a functional configuration diagram of an information processing apparatus according to the second embodiment. In FIG. 11, the same reference numerals are assigned to the same configurations as those in FIG. 3, and detailed description thereof will be omitted. As shown in FIG. 11, an information processing apparatus 20A according to the second embodiment is different from that of the first embodiment in that the information processing apparatus 20A further comprises a search unit 26 and performs a task of searching for a medical image. In the second embodiment, the analysis unit 24 performs the process of deriving the feature amount V0. Therefore, the derivation model 30 according to the second embodiment may not comprise the MLP 34.

In the second embodiment, it is assumed that a large number of medical images are saved in the image DB 6 in association with a feature amount V1. The feature amount V1 of the saved medical image is derived by a derivation model (not shown) in which machine learning is performed to derive the feature amount V1 from the medical image. The feature amount V1 of the medical image and the feature amount V0 derived by the analysis unit 24 are n-dimensional vectors distributed in the same feature space. The medical image saved in the image DB 6 is referred to as a reference image in the following description.

Further, in the information processing apparatus 20A according to the second embodiment, as in the first embodiment, the radiologist interprets the target medical image GO in the interpretation WS 3, and inputs comments on findings including the interpretation result by using the input device 15. The division unit 22 divides the input comments on findings into words, and the determination unit 23 derives a determination result of the attribute and the factuality of each word. Then, the analysis unit 24 derives a feature vector of the comments on findings as the feature amount V0 by performing the weighting calculation of the feature vector 41 of each word in the same manner as in the first embodiment.

The search unit 26 refers to the image DB 6 and searches for a reference image associated with the feature amount V1 which is close to the feature amount V0 derived by the analysis unit 24 in the feature space. FIG. 12 is a diagram for describing the search performed in the information processing apparatus 20A according to the second embodiment. In FIG. 12, the feature space is shown in two dimensions for the sake of description. Further, for the sake of description, five feature amounts V1-1 to V1-5 are plotted in the feature space.

The search unit 26 specifies a feature amount whose distance from the feature amount V0 is within a predetermined threshold value in the feature space. In FIG. 12, a circle 60 having a radius dl centered on the feature amount V0 is shown. The search unit 26 specifies a feature amount included in the circle 60 in the feature space. In FIG. 12, three feature amounts V1-1 to V1-3 are specified.

The search unit 26 searches the image DB 6 for the reference image associated with the specified feature amounts V1-1 to V1-3, and acquires the searched for reference image from the image server 5.

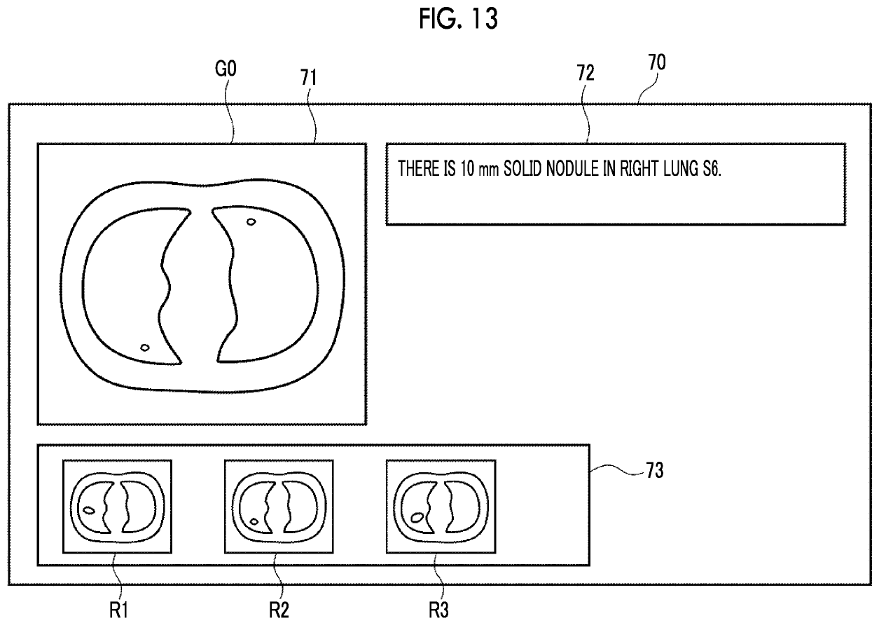

The display controller 25 displays the acquired reference image on the creation screen of the interpretation report. FIG. 13 is a diagram showing a creation screen of an interpretation report in the second embodiment. As shown in FIG. 13, a creation screen 70 includes an image display region 71, a sentence display region 72, and a result display region 73. The target medical image GO is displayed in the image display region 71. The comments on findings input by the radiologist are displayed in the sentence display region 72. In FIG. 13, the comment on findings of “There is a 10 mm solid nodule in a right lung S6” is displayed.

The reference image searched for by the search unit 26 is displayed in the result display region 73. In FIG. 13, three reference images R1 to R3 are displayed in the result display region 73.

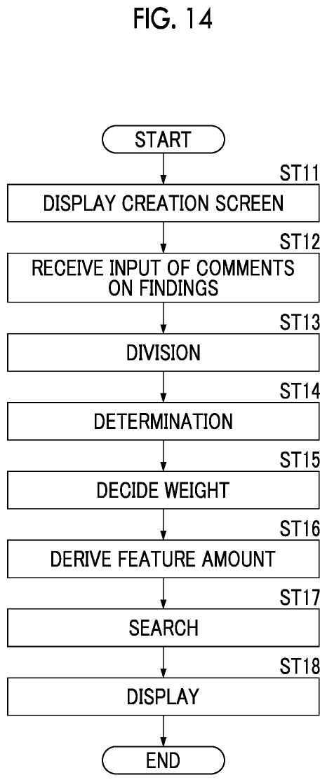

Next, a process performed in the second embodiment will be described. FIG. 14 is a flowchart showing a process performed in the second embodiment. It is assumed that the target medical image GO is acquired from the image server 5 by the information acquisition unit 21 and is saved in the storage 13.

First, the display controller 25 displays the creation screen of the interpretation report (Step ST11) and receives the input of the comments on findings (Step ST12). Next, the division unit 22 divides the comments on findings into words (Step ST13), and the determination unit 23 determines at least one of the attribute, factuality, or relationship of each divided word (Step ST14). Then, the weighting calculation mechanism 33 of the analysis unit 24 decides the weight for each word according to the determination result (Step ST15), and performs weighting calculation on the feature amount for each word based on the decided weight, thereby deriving the feature amount V0 of the comments on findings (Step ST16).

Subsequently, the search unit 26 refers to the image DB 6 and searches for a reference image associated with the feature amount V1 which is close to the feature amount V0 (Step ST17). Then, the display controller 25 displays the searched for reference image on the display 14 (Step ST18), and the process ends.

The reference images R1 to R3 searched for in the second embodiment are medical images having similar features to the comments on findings input by the radiologist. Since the comments on findings relate to the target medical image GO, the reference images R1 to R3 have similar cases to the target medical image GO. Therefore, according to the second embodiment, it is possible to interpret the target medical image GO and create an interpretation report by referring to the reference image having a similar case. Further, the interpretation report for the reference image can be acquired from the report server 7 and used for creating the interpretation report for the target medical image GO.

In the above first and second embodiments, the weight is decided according to attributes and factuality, but the present disclosure is not limited thereto. In addition to the attribute and the factuality, relationship may also be used to decide the weight. Hereinafter, this will be described as a third embodiment.

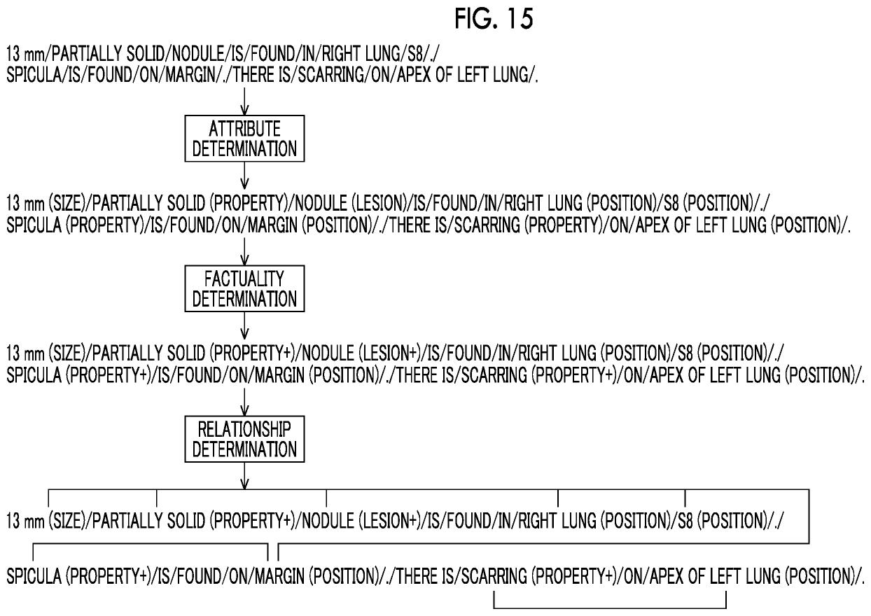

FIG. 15 is a diagram for describing the determination of an attribute, factuality, and relationship. In addition, in the third embodiment, it is assumed that the comments on findings are “A 13 mm partially solid nodule is found in a right lung S8. A spicula is found on the margin. There is scarring on the apex of the left lung.” In addition, the division unit 22 divides the comments on findings into “A 13 mm/partially solid/nodule/is found/in/a right lung/S8/. /A spicula/is found/on/the margin/. /There is/scarring/on/the apex of the left lung/.”

In the third embodiment, the determination unit 23 determines the relationship in addition to the attribute and factuality of each divided word. Regarding the above comments on findings, the determination unit 23 determines that “right lung”, “S8”, “margin”, and “apex of the left lung” have the attribute of the position, “13 mm” has the size attribute, the “partially solid”, “spicula”, and “scarring” have the property attribute, and “nodule” has the lesion attribute.

With respect to the factuality, the determination unit 23 determines that all the factualities of the words “partially solid”, “nodule”, “spicula”, and “scarring” are positive.

Regarding the relationship, the determination unit 23 derives the relationship between words. For example, among the words included in the comments on findings, the “nodule”, which is a word related to the task of specifying the disease name in the first embodiment, is associated with the word “13 mm” of the size attribute, the “right lung” and “S8” of the position attribute, and the “partially solid” and “spicula” of the property attribute”, but is not associated with the “apex of the left lung” of the position attribute and the “scarring” of the property attribute. Also, the “spicula” of the property attribute is associated with the “margin” of the position attribute, but is not associated with the “apex of the left lung” of the position attribute and the “scarring” of the property attribute. In addition, the “scarring” of the property attribute is associated with the “apex of the left lung” of the position attribute.

The relationship may be derived by referring to a table in which the presence or absence of a relationship between a large number of words is defined in advance. Further, the relationship may be derived using a derivation model constructed by performing machine learning to output the presence or absence of the relationship between words. In addition, words related to the task of specifying the disease name may be specified as keywords, and all words that qualify the keywords may be specified as related words.

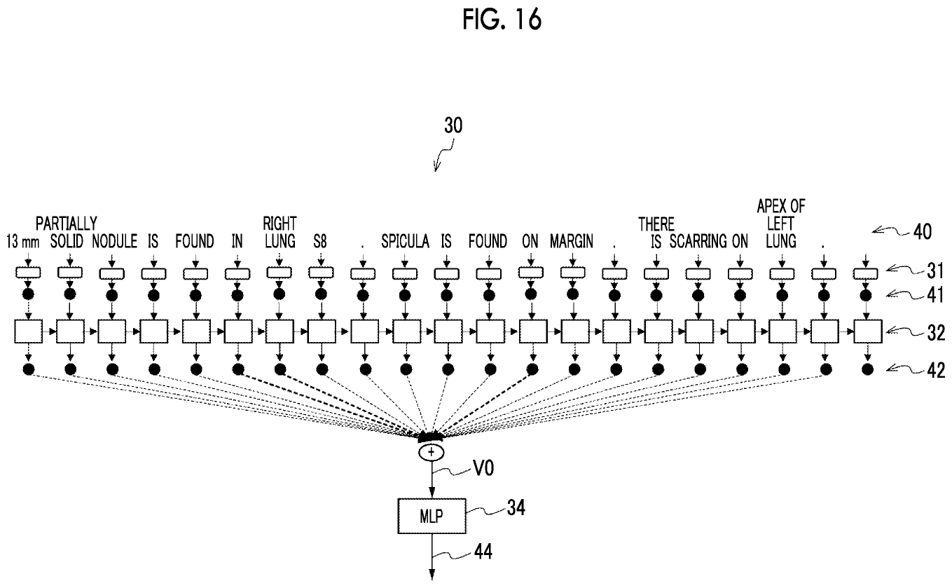

In the third embodiment, the weighting calculation mechanism 33 of the derivation model 30 specifies a word related to a word related to a task performed by the information processing apparatus, and decides a weight so that a weight for the word determined by the predetermined attribute and factuality among the specified words is greater than a weight for a word determined by an attribute and factuality other than the predetermined attribute and factuality. FIG. 16 is a diagram for describing weighting in the second embodiment. In FIG. 16, the same reference numerals are assigned to the same configurations as those in FIG. 6, and detailed description thereof will be omitted here.

Here, the word related to the word related to the task is “nodule”, and the word related to “nodule” is “13 mm”, “right lung”, “S8”, “partially solid”, and “spicula”. Therefore, in addition to the “nodule”, the weighting calculation mechanism 33 decides the weight so that the weights for the “partially solid” and “spicula” among “13 mm”, “right lung”, “S8”, “partially solid”, and “spicula” are greater than the weights for the other words. In FIG. 16, by making the arrows in the weighting calculation mechanism 33 of the feature vectors 42 of the “partially solid”, “nodule”, and “spicula” output by the RNN layer 32 thicker than the other arrows, it is shown that the weights for the “partially solid”, “nodule”, and “spicula” are greater than the weights for other words.

As in the third embodiment, by deciding the weight for each unit by using the relationship in addition to the attribute and the factuality, it is possible to derive the feature amount V0 effective for a task such as specifying a disease name represented by comments on findings.

In the first and second embodiments, the attribute and factuality of each divided word are determined, and in the third embodiment, the attribute, factuality, and relationship of each divided word are determined. However, the present disclosure is not limited thereto. Only one of the attribute, factuality, and relationship of each divided word may be determined, or only any combination of any two of these may be determined.

Further, in the first embodiment, the disease name represented by the comments on findings is specified by using a derivation model for deriving the feature amount of the comments on findings about the medical image, but the present disclosure is not limited thereto. For example, it goes without saying that the technique of the present disclosure can be applied to a task of specifying the content of a comment by using a derivation model for deriving a feature amount of a sentence such as a comment on a photographic image.

Further, in the above embodiments, for example, as hardware structures of processing units that execute various kinds of processing, such as the information acquisition unit 21, the division unit 22, the determination unit 23, the analysis unit 24, the display controller 25, and the search unit 26, various processors shown below can be used. As described above, the various processors include a programmable logic device (PLD) as a processor of which the circuit configuration can be changed after manufacture, such as a field programmable gate array (FPGA), a dedicated electrical circuit as a processor having a dedicated circuit configuration for executing specific processing such as an application specific integrated circuit (ASIC), and the like, in addition to the CPU as a general-purpose processor that functions as various processing units by executing software (programs).

One processing unit may be configured by one of the various processors, or may be configured by a combination of the same or different kinds of two or more processors (for example, a combination of a plurality of FPGAs or a combination of the CPU and the FPGA). In addition, a plurality of processing units may be configured by one processor. As an example where a plurality of processing units are configured by one processor, first, there is a form in which one processor is configured by a combination of one or more CPUs and software as typified by a computer, such as a client or a server, and this processor functions as a plurality of processing units. Second, there is a form in which a processor for realizing the function of the entire system including a plurality of processing units via one integrated circuit (IC) chip as typified by a system on chip (SoC) or the like is used. In this way, various processing units are configured by one or more of the above-described various processors as hardware structures.

Furthermore, as the hardware structure of the various processors, more specifically, an electrical circuit (circuitry) in which circuit elements such as semiconductor elements are combined can be used.

What is claimed is:

1. An information processing apparatus comprising at least one processor,
wherein the processor is configured to
divide a sentence into predetermined units,
determine at least one of an attribute, a factuality, or a relationship of each unit,
decide a weight for each unit according to a result of the determination, and
derive a feature amount of each unit by using a derivation model constructed by machine learning and derive a feature amount of the sentence by performing weighting calculation on the feature amount of each unit based on the weight.

2. The information processing apparatus according to claim 1, wherein the processor is configured to decide a weight so that a weight for a word determined by a predetermined attribute, factuality, and relationship is greater than a weight for a word determined by an attribute, factuality, and relationship other than the predetermined attribute, factuality, and relationship.

3. The information processing apparatus according to claim 2, wherein the predetermined attribute, factuality, and relationship are decided according to a task using the derived feature amount of the sentence.

4. The information processing apparatus according to claim 2, wherein the processor is configured to decide the weight by using the derivation model.

5. The information processing apparatus according to claim 1, wherein the processor is configured to display the sentence by emphasizing words determined by a predetermined attribute, factuality, and relationship.

6. The information processing apparatus according to claim 1, wherein the processor is configured to perform a task of specifying a content of the sentence by using the derived feature amount.

7. The information processing apparatus according to claim 1, wherein the processor is configured to perform a task of searching for an image corresponding to the sentence by using the derived feature amount.

8. An information processing method comprising:
dividing a sentence into predetermined units;
determining at least one of an attribute, a factuality, or a relationship of each unit;
deciding a weight for each unit according to a result of the determination; and
deriving a feature amount of each unit by using a derivation model constructed by machine learning and deriving a feature amount of the sentence by performing weighting calculation on the feature amount of each unit based on the weight.

9. A non-transitory computer-readable storage medium that stores an information processing program causing a computer to execute a procedure comprising:
dividing a sentence into predetermined units;
determining at least one of an attribute, a factuality, or a relationship of each unit;
deciding a weight for each unit according to a result of the determination; and
deriving a feature amount of each unit by using a derivation model constructed by machine learning and deriving a feature amount of the sentence by performing weighting calculation on the feature amount of each unit based on the weight.
